SEQUENCE LISTING (1 sequence)

SEQ ID NO: 1 - RNA, 1421 nt, Bacillus amyloliquefaciens Ba-BPD1
caagucgagcggacagaugggagcuugcucccugauguuagcggcggacgggugaguaacacguggguaaccugccuguaagacugggauaacuccgggaaaccggggcuaauaccggaugguuguuugaaccgcaugguucagacauaaaagguggcuucggcuaccacuuacagauggacccgcggcgcauuagcuaguuggugagguaacggcucaccaaggcgacgaugcguagccgaccugagagggugaucggccacacugggacugagacacggcccagacuccuacgggaggcagcaguagggaaucuuccgcaauggacgaaagucugacggagcaacgccgcgugagugaugaagguuuucggaucguaaagcucuguuguuagggaagaacaagugccguucaaauagggcggcaccuugacgguaccuaaccagaaagccacggcuaacuacgugccagcagccgcgguaauacguagguggcaagcguuguccggaauuauugggcguaaagggcucgcaggcgguuucuuaagucugaugugaaagcccccggcucaaccggggagggucauuggaaacuggggaacuugagugcagaagaggagaguggaauuccacguguagcggugaaaugcguagagauguggaggaacaccaguggcgaaggcgacucucuggucuguaacugacgcugaggagcgaaagcguggggagcgaacaggauuagauacccugguaguccacgccguaaacgaugagugcuaaguguuaggggguuuccgccccuuagugcugcagcuaacgcauuaagcacuccgccuggggaguacggucgcaagacugaaacucaaaggaauugacgggggcccgcacaagcgguggagcaugugguuuaauucgaagcaacgcgaagaaccuuaccaggucuugacauccucugacaauccuagagauaggacguccccuucgggggcagagugacagguggugcaugguugucgucagcucgugucgugagauguuggguuaagucccgcaacgagcgcaacccuugaucuuaguugccagcauucaguugggcacucuaaggugacugccggugacaaaccggaggaagguggggaugacgucaaaucaucaugccccuuaugaccugggcuacacacgugcuacaauggacagaacaaagggcagcgaaaccgcgagguuaagccaaucccacaaaucuguucucaguucggaucgcagucugcaacucgacugcgugaagcuggaaucgcuaguaaucgcggaucagcaugccgcggugaauacguucccgggccuuguacacaccgcccgucacaccacgagaguuuguaacacccgaagucggugagguaaccuuuuaggagccagccgccgaag

(19) United States
(12) Patent Application Publication
(10) Pub. No.: US 2015/0147303 A1
(43) Pub. Date: May 28, 2015

(54) NOVEL STRAIN OF BACILLUS AMYLOLIQUEFACIENS AND ITS USE

(71) Applicant: FENG-CHIA HSIEH, TAICHUNG CITY (TW)

(72) Inventor: FENG-CHIA HSIEH, TAICHUNG CITY (TW)

(21) Appl. No.: 14/607,838

(22) Filed: Jan. 28, 2015

Related U.S. Application Data
(63) Continuation-in-part of application No. 12/329,155, filed on Dec. 5, 2008.

(51) Int. Cl. A01N 63/00; A23L 3/3571; A61K 35/74

(52) U.S. Cl. CPC A01N 63/00; A61K 35/74; A23L 3/3571; A23V 2002/00
USPC 424/93.46

(57) ABSTRACT

An isolated Bacillus amyloliquefaciens Ba-BPD1 having an Accession No. of DSM 21836 is provided. This novel strain has unique 16S ribosomal RNA sequenced as SEQ ID NO:1 and produces amylase, protease, cellulase and lipase, fibrinolytic enzyme to show their biodegradation capacities. Further, B. amyloliquefaciens Ba-BPD1 produces the antibiotic substances, such as iturin, fengycin and surfactin, and has antimicrobial capacity for inhibiting the fungal or bacterial growth. In conclusion, the novel strain of Bacillus amyloliquefaciens Ba-BPD1 and its products can be applied in agriculture, wastewater treatment, food industry and chemical industry.

### FIELD OF THE INVENTION

The present invention relates to a novel strain of Bacillus amyloliquefaciens. In particular, the present invention relates to a novel strain of B. amyloliquefaciens Ba-BPD1 or a mutant thereof for producing multiple enzymes, multiple antibiotic substances, and biosurfactants.

### BACKGROUND OF THE INVENTION

Microorganisms and products generated therefrom have widely applied in improving human lives, such as food, beverage, pharmaceuticals, chemical industries and agriculture, etc. These applications enormously decrease the production and/or treatment cost and satisfy humans' demands.

Some microorganisms produce enzymes to decompose macromolecules. For instance, Yarrowia lipolytica produces lipase applied in decreasing the chemical oxygen demand (COD) level in the olive mill wastewater treatment (Lanciotti et al., 2005). Pseudomonas aerugenosa produces alkaline protease to hydrolyze animal fleshing, the major proteinaceous solid waste from the leather manufacturing industries (Kumar et al., 2008). Aspergillus terreus produces carboxymethyl cellulase (CMCase) to biodegrade the lignocellulosic waste (Emtiazi et al., 2001). However, these bacteria were proved that their key enzyme functions in decomposing one substrate. While treating with the more complicated components of municipal wastewater, adding various microorganisms to decompose various organic macromolecules is necessary and inevitable. The cost will be increased, the economic benefit will be decreased, and the treatment process will be more complicated. Further, these supplemented bacteria might be competed the growth and the predominance with each other. If one bacterial strain has multiple-enzyme-producing activity but only utilizes in one category, the economic value of this bacterial strain is less than that of another bacterial strain having multiple-enzyme-producing activity and utilizing in more categories.

In addition to the biodegradation of organic waste, microorganisms also produce antibiotic substances to antagonize with other fungi and bacteria. Generally speaking, antibiotics are produced or extracted from fungi and are applied in the medicine and pharmacology. However, plants, fruits and animals also face the fungal or bacterial infection while maturing and living. The traditional fungicides, bactericides and chemical synthetic agents not only eliminate the fungal and bacterial infections, but also endanger humans and the environment. If bacterial strains are founded to produce biologically antibiotics to inhibit the fungal or bacterial growth, these strains will be beneficial in agriculture and livestock industry.

Recently, biosurfactants, a unique class of amphiphilic biological compounds produced by microorganisms, have been shown to have a variety of potential applications in the remediation of organic- and metal-contaminated sites (Bodour et al., 2003). Biosurfactants can reduce surface tension, stabilize emulsion and promote foaming, and are generally non-toxic and biodegradable. Biosurfactants are grouped into two major classes, glycolipids and lipoproteins, wherein lipoproteins include iturin, surfactin and fengycin, etc., which are produced only by Bacillus sp. Biosurfactant producing microorganisms may play an important role in the accelerated bioremediation of hydrocarbon contaminated sites. Biosurfactant can also be used in the enhanced oil recovery and may be considered for other potential applications in the environmental protection. Other applications include herbicides and pesticides formulations, detergents, health care and cosmetics, pulp and paper, coal, textiles, ceramic processing and food industries, uranium ore-processing and mechanical dewatering of peat.

Accordingly, if a microorganism is found to produce multiple enzymes and molecules, it will be beneficial on human's life and economy. The isolated Bacillus amyloliquefaciens Ba-BPD1 has the potential to produce the above-mentioned enzymes, antibiotics, and biosurfactants while comparing with the other microorganisms.

It is therefore attempted by the applicant to deal with the above situation encountered in the prior art.

### SUMMARY OF THE INVENTION

In accordance with one aspect of the present invention, an isolated microorganism having a specific 16S ribosomal RNA (rRNA) sequence is provided and classified as a strain of Bacillus amyloliquefaciens Ba-BPD1, which is nominated as an Accession No.: DSM 21836. This novel bacterial strain produces specific and useful enzymes, such as lipase for decomposing fat, amylase for hydrolyzing starch, cellulase for hydrolyzing cellulose and protease for hydrolyzing protein.

Because organic components exist in the wastewater, the novel multiple-enzyme-producing B. amyloliquefaciens Ba-BPD1 is applied in processing wastewater, plumbing system, animal feed and kitchen waste, so as to improve the decomposition of organic components in the sewage, and the quality and efficiency of the wastewater and garbage treatment processes. Therefore, this novel bacterial strain and enzymes produced therefrom can be manufactured as the detergent and applied in decontamination and food manufacture.

Further, the multiple-enzyme-producing B. amyloliquefaciens Ba-BPD1 is applied in the agriculture, including silage inoculants, livestock manure treatment and Direct Fed Microbials in livestock feed formulations.

Further, the isolated B. amyloliquefaciens Ba-BPD1 can promote plant growth because of the enzyme activities of decomposing the macromolecules into basic organic molecules.

Furthermore, the fibrinolytic enzyme produced by B. amyloliquefaciens Ba-BPD1 can hydrolyze thrombus, so as to decrease the amount of fibrin clots in the blood, prevent and cure cardiovascular disease, thrombosis, arteriosclerosis, endometriosis and cancer. Therefore, fibrinolytic enzyme can improve the health in human and animals.

In addition, the isolated B. amyloliquefaciens Ba-BPD1 produce antibiotic substances (such as iturin, surfactin and fengycin) and the surfactant. In particular, iturin means iturin A and iturin A homologues. Iturin, surfactin and fengycin belong to lipopeptide and are beneficial in preventing and treating fungal and/or bacterial infection, and these infections are infected to plants, animals and fruits.

In addition, B. amyloliquefaciens Ba-BPD1 produces biosurfactants, including surfactin, iturin and fengycin, to inhibit the growths of plant pathogens and animal pathogens, and has potential in the antibiotic production.

Another object of the present invention is to provide the novel bacteria strain, B. amyloliquefaciens Ba-BPD1, and/or antibiotic substances produced from such bacteria for use as an antifungal agent, and to suppress the growth of at least one microorganism which belongs to at least one genus of fungi selected from the group consisting of Botrytis, Colletotrichum, Rhizoctonia, Fusarium, Sclerotium, Alternaria, Phytophthora, Aspergillus, Penicillium, Pestalotiopsis, and Botryodiplodia.

Among these, the fungal infection results from one fungus selected from a group consisting of Botrytis elliptica, Botrytis cinerea, Glomerella cingulata, Colletotrichum musae, Colletotrichum gloeosporioides, Rhizoctonia solani, Fusarium oxysporum f. sp. pisi, Fusarium oxysporum f. sp. lycopersici, Fusarium solani, Sclerotium rolfsii Saccardo, Alternaria mali, Phytophthora capsici, Aspergillus niger, Penicillium italicum, Pestalotiopsis eugeniae and Botryodiplodia theobromae.

Another object of the present invention is to provide B. amyloliquefaciens Ba-BPD1 and/or antibiotic substances produced from such bacteria for use as an antibacterial agent, and to suppress the growth of at least one microorganism which belongs to at least one genus of bacteria selected from the group consisting of Erwinia, Acidovorax, Agrobacterium, Burholderia, Enterobactor, Pseudomonas, Ralstonia, Xanthomonas, Bacillus, and Salmonella. Among these, the bacterial infection results from one bacterium selected from a group consisting of Erwinia chrysanthemi, Erwinia carotovora subsp. carotovora, Acidovorax avenae subsp. citrulli, Agrobacterium tumefaciens, Burholderia caryophylli, Enterobactor cloaceae, Pseudomonas syringae, Ralstonia solanacearum, Xanthomonas axonopodis pv. cirti, Xanthomonas axonopodis pv. vesicatoria, Xanthomonas campestris pv. compestris, Xanthomonas oryzae pv. oryzae, Bacillus cereus and Salmonella.

Further, the isolated B. amyloliquefaciens Ba-BPD1 is being an antimicrobial agent for suppressing the fungal and/or the bacterial growth. The antifungal agent inhibits one fungus selected from a group consisting of Botrytis elliptica, Botrytis cinerea, Glomerella cingulata, Colletotrichum muscle, Colletotrichum gloeosporioides, Rhizoctonia solani, Fusarium oxysporum f. sp. pisi, Fusarium oxysporum f. sp. lycopersici, Fusarium solani, Sclerotium rolfsii Saccardo, Alternaria mali, Phytophthora capsici, Aspergillus niger, Penicillium italicum, Pestalotiopsis eugeniae and Botryodiplodia theobromae. The antibacterial agent inhibits one bacterium selected from a group consisting of Erwinia chrysanthemi, Erwinia carotovora subsp. carotovora, Acidovorax avenae subsp. citrulli, Agrobacterium tumefaciens, Burholderia caryophylli, Enterobactor cloaceae, Pseudomonas syringae, Ralstonia solanacearum, Xanthomonas axonopodis pv. cirti, Xanthomonas axonopodis pv. vesicatoria, Xanthomonas campestris pv. compestris, Xanthomonas oryzae pv. oryzae, Bacillus cereus and Salmonella.

Surfactin inhibits the pathogenic growth of the livestock and food, and prevents and/or treats animals or plants infected from pathogens. Additionally, because of the characteristics of non-toxicity to the environment and the better biodegradation, surfactin is widely applied in detergent, cosmetics, food, pharmaceuticals, petroleum industry, agriculture and environmental protection, etc.

Preferably, the isolated B. amyloliquefaciens Ba-BPD1 is applied as whole broth culture, supernatant, wettable powders, granules, water dispersible granules, suspension concentrate (flowable concentrate) and microencapsulations.

In accordance with another aspect of the present invention, an isolated mutant of B. amyloliquefaciens Ba-BPD1 with an Accession No.: DSM 21836 has a specific 16S ribosomal RNA sequenced as SEQ ID NO:1.

In accordance with another aspect of the present invention, a composition includes an isolated microorganism of strain of B. amyloliquefaciens Ba-BPD1 having an Accession No.: DSM 21836.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is an analytic pattern of Iturin A and surfactin produced from B. amyloliquefaciens Ba-BPD1 by liquid chromatography/time-of-flight-mass spectrometry (LC/TOF-MS) in accordance with the seventh and eighth embodiments of the present invention; and

FIG. 2 is an analytic pattern of fengycin produced from B. amyloliquefaciens Ba-BPD1 by LC/TOF-MS in accordance with the seventh embodiment of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

### Embodiment 1

### Characteristics of the Novel Strain of Bacillus amyloliquefaciens Ba-BPD1

The novel strain of B. amyloliquefaciens Ba-BPD1 was isolated from the soil in Lishan, Taichung County, Taiwan by the inventors, and B. amyloliquefaciens Ba-BPD1 was further incubated, identified and preserved. While incubating B. amyloliquefaciens Ba-BPD1, one single colony thereof was inoculated and incubated overnight in 6 ml of Luria-Bertani (LB, Miller; Difco) broth. The cultured broth was then inoculated in 500 ml of LB broth at a ratio of 1:100, and the inoculated broth was further incubated at 30° C. at 150 rpm for 6 days.

Furthermore, B. amyloliquefaciens Ba-BPD1 has a specific 16S ribosomal RNA (rRNA) sequence in comparison with other bacteria. The partial 16S rRNA sequence was sequenced and the GenBank accession number was nominated as EF137183, which will be published on the website of the National Center for Biotechnology Information (http://www.ncbi.nlm.nih.gov/Genbank/) on Dec. 31, 2009. The partial 16S rRNA sequence named as SEQ ID NO:1 as follows becomes the distinctive and significant characteristic of B. amyloliquefaciens Ba-BPD1.

Bacillus amyloliquefaciens Ba-BPD1 was deposited in the Deutsche Sammlung von Mikroorganismen and Zellkulturen GmbH (DSMZ), Inhoffenstr. 7B, D-38124 Braunschweig, Germany, on Sep. 11, 2008, under the rules of Budapest Treaty, and the deposit number was DSM 21836.

### Embodiment 2

### Production of Amylase from B. amyloliquefaciens Ba-BPD1

In order to prove that B. amyloliquefaciens Ba-BPD1 can produce amylase to hydrolyze starch, an amylase hydrolysis test was performed as follows. A single colony of B. amyloliquefaciens Ba-BPD1 was picked from the nutrient agar (NA) plate and mixed with 50 μl of distilled water to be the bacterial medium. Then, 50 μl of the bacterial medium was dripped on a 1-cm-diameter disc which was further stuck on a yeast extract-soluble starch agar (YSA) plate (containing 1.0% of yeast extract, 1.0% of soluble starch and 1.5% of agar). This YSA plate was incubated at 30° C. for 2 to 3 days. After incubation, 3 to 4 ml of iodine solution (containing 0.3% (w/v) of iodine and 3% (w/v) of potassium iodine) was immersed on the YSA plate. The colony size and the clear zone formation of B. amyloliquefaciens Ba-BPD1 were measured within 5 minutes. The blue-black color shown on the medium means that starch is not hydrolyzed; however, the clear zone surrounding the colony means starch is hydrolyzed. It was found that the colony size in diameter and the clear zone in diameter were 1.57 cm and 2.81 cm respectively in triplet.

Ito et al. (1998) also proved that the alkaliphilic Bacillus strains produce the alkaline extracellular detergent enzyme, including α-amylase, to apply in the heavy-duty power detergents and the automatic dishwasher detergents, so as to decompose starch in the wastewater. Therefore, amylase produced from B. amyloliquefaciens Ba-BPD1 can be applied in hydrolyzing starch in the wastewater, waste, agriculture industry, and food industry.

### Embodiment 3

### Production of Protease from B. amyloliquefaciens Ba-BPD1

In order to prove that B. amyloliquefaciens Ba-BPD1 produce protease to hydrolyze protein, an proteolytic test was performed as follows. The bacterial medium of B. amyloliquefaciens Ba-BPD1 was prepared as Embodiment 2. Fifty (50) μl of the bacterial medium thereof was dripped on a 1-cm-diameter disc which was further disposed on a skim milk agar (SMA) plate (containing 1.5% of skim milk, 1.3% of nutrient broth and 1.5% of agar) (Elsheikh et al., 1986). This SMA plate was incubated at 30° C. for 2 to 3 days, and the colony size and the clear zone formation of B. amyloliquefaciens Ba-BPD1 were calculated. The clear zone surrounding the colony means protein in the skim milk is hydrolyzed by the bacterium. It was found that the colony size in diameter and the clear zone in diameter were 1.77 cm and 3.61 cm respectively in triplet.

Kumar et al. (2008) found that Pseudomonus aeruginosa produce the alkaline protease to hydrolyze the proteinaceous solid waste generated from the leather manufacturing industries. In addition, Drouin et al. (2008) proved that protease produced from Bacillus licheniformis perform the proteolytic activity on the wastewater sludge. In Embodiment 3, protease produced from B. amyloliquefaciens Ba-BPD1 also can be applied in hydrolyzing protein in the wastewater, waste, agriculture industry, food industry, and be prepared as the component of detergent or laundry detergent.

### Embodiment 4

### Production of Cellulase from B. amyloliquefaciens Ba-BPD1

In order to prove that B. amyloliquefaciens Ba-BPD1 produce cellulase to decompose cellulose, an cellulase hydrolysis test was performed as follows. The bacterial medium of B. amyloliquefaciens Ba-BPD1 was prepared as Embodiment 2. Fifty (50) μl of the bacterial medium thereof was dripped on an 1-cm-diameter disc which was further disposed on a Mandel-Reese (M-R) agar plate (containing 1.0% of carboxyl methyl cellulose (CMC), 0.1% of peptone, 0.03% of urea, 0.14% of (NH₄)₂SO₄, 0.2% of KH₂PO₄, 0.04% of CaCl₂.H₂O, 0.03% of MgSO₄.7H₂O, 5×10⁻⁴% of FeSO₄.7H₂O, 1.4×10⁻³% of ZnSO₄.7H₂O, 1.6×10⁻³% of MnSO₄.4H₂O, 2×10⁻⁴% of CoCl₂.6H₂O and 1.5% of agar; adjusting pH to 6.0 and autoclaving) (Mandel and Reese, 1960). After this M-R agar plate was incubated at 30° C. for 2 days, 3 to 4 ml of 0.1% of Congo red was immersed thereon for 30 minutes. The un-conjugated Congo red was washed with 1 M of NaCl. Congo red forms agglomerates or colloids by hydrogen bonding and then conjugates with the cellulose, and clear zone formation means cellulose without bonded with Congo red. It was found that the clear zone in diameter formed by B. amyloliquefaciens Ba-BPD1 was 2.3 cm in triplet.

Alam et al. (2008) proved that Trichoderma harzianum produce cellulase for hydrolyzing cellulose in the bioconversion of sewage sludge. Sangave and Pandit (2006) also published that cellulase was utilized in the pretreatment step in the biodegradability of distillery wastewater, so as to transform cellulose into the simple biological molecules. Because the main component of M-R agar plate is carboxyl methyl cellulose, it is obvious that B. amyloliquefaciens Ba-BPD1 can produce cellulase to digest cellulose and represent the clear zone formation while digesting. Therefore, B. amyloliquefaciens Ba-BPD1 have significant economic value on treating cellulose in the sewage water because of the cellulase production and hydrolysis capability. It is believed that the brand-new bacterial strain, B. amyloliquefaciens Ba-BPD1, can produce cellulase to hydrolyze cellulose in the waste treatment.

### Embodiment 5

### Production of Lipase from B. amyloliquefaciens Ba-BPD1

In order to prove that B. amyloliquefaciens Ba-BPD1 produce lipase to decompose lipid, an lipase hydrolysis test was performed as follows. First, a single colony of B. amyloliquefaciens Ba-BPD1 was inoculated into 5 ml of nutrient broth (NB), which was further incubated at 30° C. at 150 rpm for 1 day. Then, 5 μl of the incubated medium was dripped on a Rhodamine B agar plate (containing 1% of olive oil, 0.001% of Rhodamine B and 1.5% of nutrient agar) and was further incubated at 30° C. for 7 days. Rhodamine B, being a dye, is incorporated into lipid as the fluorescent marker in biotechnology applications such as fluorescence microscopy. The clear zone represents that the lipid is hydrolyzed and Rhodamine B cannot be incorporated thereinto. Accordingly, it was found that the colony of B. amyloliquefaciens Ba-BPD1 showed fluorescence and the clear zone surrounding the colony in diameter was 0.6 cm.

Ertu\check{g}rul et al. (2007) published that lipase produced from Bacillus sp. showed its lipase activity on decomposing the compositions of the olive mill wastewater, triacetin, Tween 80 and whey, etc. Even, the immobilized lipase was utilized in the hydrolysis of wastewater with high oil and grease concentration (Jeganathan et al. 2007). Therefore, lipase produced from B. amyloliquefaciens Ba-BPD1 can be applied in the lipid degradation of wastewater, waste, agriculture industry, and food industry.

### Embodiment 6

### Production of Fibrinolytic Enzyme from B. amyloliquefaciens Ba-BPD1

Fibrin is a critical blood component responsible for hemostasis, which has been used extensively as a versatile biopolymer scaffold in tissue engineering. Fibrin alone or in combination with other materials (such as fibrinogen and thrombin) has been used as a biological scaffold for stem or primary cells to regenerate adipose tissue, bone, cardiac tissue, cartilage, liver, nervous tissue, ocular tissue, skin, tendons, and ligaments, and shows a great potential in the tissue regeneration and wound healing (Ahmed et al., 2008). However, if fibrin accumulates as fibrin clots in the blood vessels or the heart, it will induce cardiovascular diseases or people will die (Hua et al., 2008). It is proved that a Bacillus sp. strain produce the fibrinolytic enzyme to be able to degrade fibrin clots (thrombus) either by forming active plasmin from plasminogen or by the direct fibrinolysis. Therefore, the fibrinolytic enzyme produced from microorganisms have a great potential in the tissue regeneration, wound healing, and life saving.

In the present invention, one single colony of B. amyloliquefaciens Ba-BPD-1 was inoculated in 5 ml of NB at 30° C. for 12 hours. After 100 μl bacterial broth from 5 ml of NB cultured broth was centrifuged, 20 μl of supernatant was dripped into the shallow hole, which was dug by a tip, of the fibrin agar plate. The fibrin agar plate then was incubated at 37° C. for 12 hours, and the formation of clear zone was observed. The result was shown that the diameter of clear zone was 1.8 cm, and demonstrated that B. amyloliquefaciens Ba-BPD-1 has ability on producing fibrinolytic enzyme to hydrolyze thrombus, and involving in the pathological situations, such as thrombosis, arteriosclerosis, endometriosis and cancer.

### Embodiment 7

### Productions of Iturin and Fengycin from B. amyloliquefaciens Ba-BPD1

Iturin A, one of the biosurfactants, is an antifungal lipopeptide to be a bioactive microbial secondary metabolite and shows attractive antibiotic properties (Hsieh et al., 2008). Iturin A produced from Bacillus sp. forms the complex with sterol molecules on the cellular membrane of pathogenic fungi (such as Rhizoctonia solani), so as to increase the size of ion-conducting channel, change the osmosis of membrane, and further leads the decomposition of mycelia of pathogenic fungi and inhibits the spore germination. Therefore, the effect on the suppression of plant pathogens is achieved. Accordingly, iturin A and Bacillus sp. are applied in the preservation of feed and/or food, the prevention and/or treatment of animals and plants, being the surfactant (or the biosurfactant) in the biodegradation and clearance in the industry, agriculture, environment, and as the antibiotic of the animal and/or plant infection (Mizumoto et al., 2007).

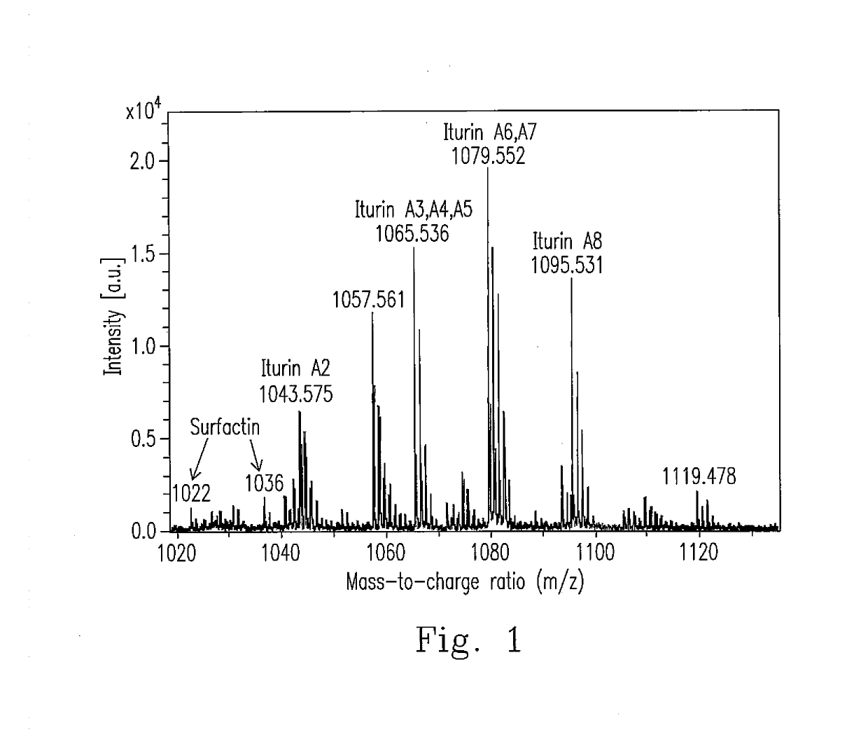

Please refer to FIG. 1, which is the analytic pattern of Iturin A produced from B. amyloliquefaciens Ba-BPD1 by liquid chromatography/time-of-flight-mass spectrometry (LC/TOF-MS) in accordance with the seventh embodiment of the present invention. In FIG. 1, the molecular weights of iturin A homologues (A2 to A8) were identified as 1043, 1057, 1065, 1079, 1095 and 1119 Da. It was shown that these iturin A homologues and B. amyloliquefaciens Ba-BPD1 can be applied in the food industry and agriculture.

Fengycin is another biologically active lipopeptide and antifungal substance produced from Bacillus subtilis and plays a major role in the antagonism of B. subtilis toward the cucurbit powdery mildew, Podosphaera fusca (Deleu et al., 2008; Romero et al., 2007). Like iturin A, fengycin also can be applied in the preservation of feed and/or food, being the surfactant (or the biosurfactant) in the biodegradation and clearance in the industry, agriculture, environment, and the prevention and/or treatment of animals and plants.

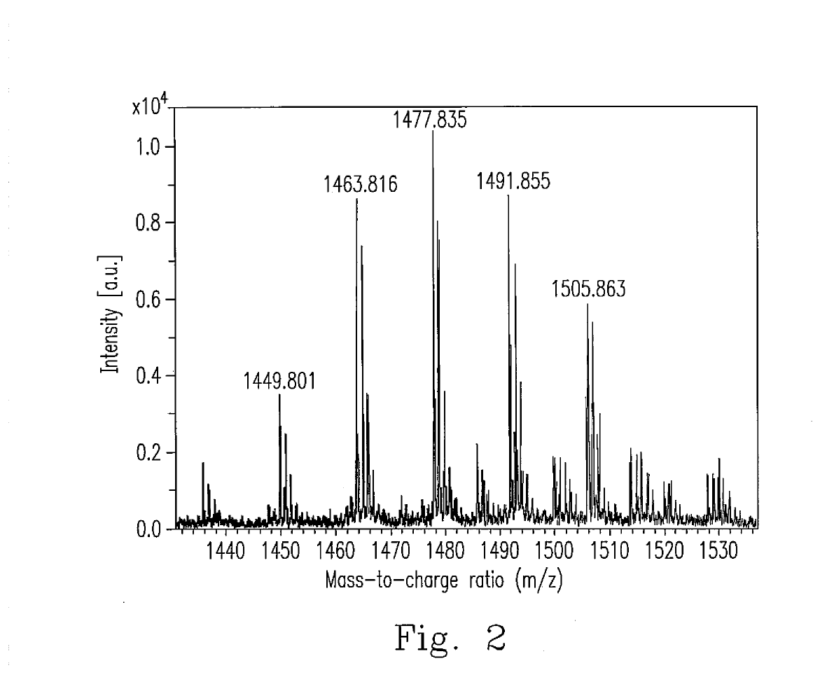

Please refer to FIG. 2, which is the analytic pattern of fengycin produced from B. amyloliquefaciens Ba-BPD1 by LC/TOF-MS in accordance with the seventh embodiment of the present invention. In FIG. 2, the molecular weights of fengycin homologues were identified as 1449, 1463, 1477, 1491 and 1505 Da. It was shown that these fengycin homologues and B. amyloliquefaciens Ba-BPD1 can be applied in the food industry and agriculture.

### Embodiment 8

### Production of Surfactin from B. amyloliquefaciens Ba-BPD1

Surfactin is a bacterial cyclic lipopeptide or surfactant being an antibiotic substance. Its amphiphilic property helps this substance to survive in both hydrophobic and hydrophilic environment. For instance, surfactin can present its antimicrobial property to Escherichia coli in milk, so as to sterilize milk (Huang et al., 2008). Whang et al. (2008) proved that surfactin has biodegradation ability of diesel-contaminated water and soil. Therefore, surfactin can be the sterilizer in food manufacturing and food preservation, and being the surfactant (or biosurfactant) in biodegradation and clearance in industry, agriculture and environment.

In order to prove that B. amyloliquefaciens Ba-BPD1 produce surfactin, a large-scaled B. amyloliquefaciens Ba-BPD1 bacterial medium and surfactin were prepared as follows. B. amyloliquefaciens Ba-BPD1 was incubated at 30° C. at 200 rpm for 16 hours, then the cultured broth was inoculated into the Cooper's medium at the ratio of 1:100 and incubated at 30° C. for 120 hours. The Cooper's medium is composed of 4% of glucose in the mineral salts (containing 0.05 M of NH₄NO₃, 0.03 M of KH₂PO₄, 0.04 M of Na₂HPO₄, 8.0×10⁻⁴ M of MgSO₄, 7.0×10⁻⁶ M of CaCl₂, 4.0×10⁻⁶ M of FeSO₄ and 4.0×10⁻⁶ M of Na₂ ethylenediaminetetraacetic acid (Na₂ EDTA)).

Crude surfactin was isolated by adding concentrated hydrochloric acid to the cultured broth of B. amyloliquefaciens Ba-BPD1 after removing the biomass by centrifugation. A precipitate was formed at pH 2 by collecting, drying and extracting with dichloromethane. This solvent was removed under the reduced pressure to obtain an off-white solid. Further purification was achieved by re-crystallization. The dichloromethane extract was dissolved in distilled water containing sufficient NaOH to achieve pH 8. This solution was further filtered through Whatman No. 1 filter paper and was titrated to pH 2 with concentrated hydrochloric acid. The white solid pellet was collected after centrifugation. In addition, authentic surfactin was purchased from Sigma (Steinheim, Germany) or was purified from culture supernatants of Bacillus spp. to be the calibration standard.

The isolated surfactin pellet was dissolved in 1 ml of methanol followed by charcoal treatment and was passed through a 0.22-μm-pore sized filter. The filtrate was subjected to the high-performance liquid chromatography (HPLC) on a reversed-phase column (RP-18, 5 μm, 4×250 mm; Merck). The column was eluted at a flow rate of 1.0 ml/min with 3.8 mM of acetonitrile-trifluoroacetic acid (80:20, v/v) and was monitored at 210 nm. The concentration of surfactin was determined with a calibration curve made with the authentic surfactin purchased from Sigma, and the total amount of 6 isoforms of surfactin were used as the concentration of surfactin. It was found that the concentration of the isolated surfactin produced from B. amyloliquefaciens Ba-BPD1 was 460 mg/L.

Please refer to FIG. 1, which is the analytic pattern of surfactin produced from B. amyloliquefaciens Ba-BPD1 by LC/TOF-MS in accordance with the eighth embodiment of the present invention. In FIG. 1, the molecular weights of surfactin homologues were identified as 1022 and 1036 Da. Therefore, surfactin produced from B. amyloliquefaciens Ba-BPD1 can be applied in food sterilization, food preservation, biodegradation and clearance in industry, agriculture and environment.

### Embodiment 9

### Antagonistic Assay Between B. amyloliquefaciens Ba-BPD1 and the Pathogenic Fungi

In accordance with the results of Embodiments 7 and 8, it was known that three lipopeptides, iturin A, fengycin and surfacin can inhibit the pathogenic fungal and bacterial growths. In order to identify the anti-pathogenic fungus ability of B. amyloliquefaciens Ba-BPD1, the antagonistic culture and assay were performed. The antagonistic assay reveals the growth inhibition of one organism to another organism.

B. amyloliquefaciens Ba-BPD1 and 21 fungi were incubated. A single colony of B. amyloliquefaciens Ba-BPD1 was inoculated into 5 ml of LB broth and incubated at 30° C. at 200 rpm for 7 days. The 1-cm-diameter mycelial disc of each fungus was stuck on the center of each potato dextrose agar (PDA) plate, which was then incubated at 25° C. to 100% confluence, and a total of 21 fungi were incubated.

While performing the antagonistic assay, an abovementioned incubated 1-cm-diameter disc of each fungus was stuck on the center of one PDA plate, and three 9-mm-diameter filters were stuck on this PDA plate. Each filter was disposed at a distance of 1.8 cm with the edge of the fungus disc, and three filters disposed on the PDA plate looked like three apexes of an equilateral triangle. Being the experimental group, 30 μl of B. amyloliquefaciens Ba-BPD1 cultured broth was dripped on each filter of one PDA plate. Being the control group, 30 μl of distilled water was dripped on each filter of another PDA plate. These PDA plates were incubated at 25° C. to 100% confluence. The confront culture between B. amyloliquefaciens Ba-BPD1 and fungus were recorded, and the inhibition distance between the B. amyloliquefaciens Ba-BPD1 disc and the fungus disc was calculated (formed crescents of inhibition around discs).

Please refer to Table 1, which is the average inhibition distance between the B. amyloliquefaciens Ba-BPD1 disc and the fungus disc in accordance with the ninth embodiment of the present invention. The longer inhibition distance is, the better inhibition effect is. In Table 1, it was shown that these 21 fungal growths could be effectively inhibited by B. amyloliquefaciens Ba-BPD1, wherein the longest inhibition distance was between B. amyloliquefaciens Ba-BPD1 and Penicillium italicum (abbreviated as Pi13) (13.5 mm), and the shortest one was between B. amyloliquefaciens Ba-BPD1 and Glomerella cingulata (abbreviated as Gc) (3.1 mm). Accordingly, the effect of growth inhibition of B. amyloliquefaciens Ba-BPD1 to fungi can be proved.

TABLE 1
The average inhibition distance between B. amyloliquefaciens Ba-
BPD1 disc and the fungus disc

| | | Average |
|---|---|---|
| | | inhibition |
| Species | Abbreviation | distance (mm) |
| Botrytis elliptica | Be | 9.2 |
| Botrytis cinerea | Bc | 8.8 |
| Glomerella cingulata | Gc | 3.1 |
| Colletotrichum musae | Cm | 9.8 |
| Rhizoctonia solani | Rs | 4.0 |
| Fusarium oxysporum f. sp. pisi | F307 | 10.5 |
| Fusarium oxysporum f. sp. lycopersici | F308 | 5.2 |
| Fusarium oxysporum f. sp. lycopersici | Fol-33 | 7.7 |
| Fusarium solani | FSO | 7.3 |
| Fusarium solani | FSL | 7.5 |
| Sclerotium rolfsii Saccardo | Sr | 3.0 |
| Alternaria mali | Am | 8.0 |
| Phytophthora capsici | PcS1 | 5.0 |
| Aspergillus niger | An12 | 5.0 |
| Aspergillus niger | An22 | 4.0 |
| Penicillium italicum | Pi13 | 13.5 |
| Penicillium italicum | Pi28 | 12.3 |
| Colletotrichum gloeosporioides | Cg-T4018 | 7.8 |
| Colletotrichum gloeosporioides | Cg-T4044 | 9.4 |
| Pestalotiopsis eugeniae | Pe | 7.3 |
| Botryodiplodia theobromae | Bot | 9.3 |

### Embodiment 10

### Antagonistic Assay Between B. amyloliquefaciens Ba-BPD1 and the Pathogenic Bacteria

Another antagonistic experiment between B. amyloliquefaciens Ba-BPD1 and the pathogenic bacteria was performed as follows. First, 60 μl of tested B. amyloliquefaciens Ba-BPD1 was dripped on a disc, which was stuck on an NA plate and incubated at 30° C. for 24 hours. Each tested pathogenic bacterium was then sprayed uniformly on each cultured B. amyloliquefaciens Ba-BPD1 agar plate, which was incubated at 30° C. for another 24 hours. Each pathogenic bacterium was tested in triplet. The inhibition zone of B. amyloliquefaciens Ba-BPD1 to each pathogenic bacterium was determined.

Please refer to Table 2, which is the inhibition zone of B. amyloliquefaciens Ba-BPD1 to each pathogenic bacterium in accordance with the tenth embodiment of the present invention. In Table 2, it was shown that the these pathogenic bacterial growths could be effectively inhibited by B. amyloliquefaciens Ba-BPD1. Therefore, it is proved that the pathogenic plant and fruit diseases caused by these bacteria could be prevented, treated or controlled. In addition, the growths of Bacillus cereus JSR01 and Salmonella TA100 could be inhibited by B. amyloliquefaciens Ba-BPD1, and the bacterial food poisoning caused thereby could be prevented and cured by B. amyloliquefaciens Ba-BPD1.

TABLE 2
The inhibition zone of B. amyloliquefaciens Ba-BPD1 to the
pathogenic bacteria

| | | Inhibition zone |
|---|---|---|
| Bacterium | Disease | in diameter (cm) |
| Acidovorax avenae subsp. citrulli | Bacterial fruit blotch of | 3.4 |
| | melon | |
| Agrobacterium tumefaciens | Crown gall | 2.3 |
| Burholderia caryophylli | Bacterial wilting | 3.5 |
| Enterobactor cloaceae | Bacterial basal rot | 2.5 |
| Erwinia carotovora subsp. carotovora | Soft rot disease | 2.3 |
| Erwinia chrysanthemi | Soft rot disease | 3.1 |
| Pseudomonas syringae | Bacterial leaf spots | 3.1 |
| Ralstonia solanacearum | Bacterial wilting | 2.9 |
| Xanthomonas axonopodis pv. cirti | Cirtus canker | 4.5 |
| Xanthomonas axonopodis pv. vesicatoria | Bacterial spot of tomato | 4.5 |
| Xanthomonas compestris pv. compestris | Black rot of brassica | 4.5 |
| Xanthomonas oryzae pv. oryzae | Bacterial leaf blight | 3.2 |
| Bacillus cereus JSR01 | Bacterial food poisoning | 0.9 |
| Salmonella TA100 | Bacterial food poisoning | 1.1 |

### Embodiment 11

### The Bacterial Inhibition Assay Between B. amyloliquefaciens Ba-BPD1 to the Aquatic Pathogenic Bacteria

The bacterial inhibition assay reveals the growth inhibition of B. amyloliquefaciens Ba-BPD1 to aquatic pathogenic bacteria. In order to identify the result of growth inhibition of aquatic pathogenic bacteria, the bacterial inhibition assay is performed as follows.

The selected aquatic pathogenic bacteria for the bacterial inhibition assay, their culture condition and specific culture media are listed as follows:

1. Aeromonas hydrophila subsp. hydrophila, 30° C., Nutrient agar
2. Edwardsiella tarda, 37° C., Nutrient agar
3. Enterococcus faecalis (also named Streptococcus faecalis), 37° C., Brain heart infusion agar
4. Enterococcus faecium, (also named Streptococcus faecium), 37° C., Brain heart infusion agar
5. Lactococcus garvieae, 30° C., Brain heart infusion agar
6. Photobacterium damselae subsp. damselae, 26° C., Marine agar
7. Streptococcus iniae, 37° C., Tryptic soy agar
8. Vibrio parahaemolyticus, 37° C., Marine agar

B. amyloliquefaciens Ba-BPD1 was incubated in Luria-Bertani broth (LB, 25 g Luria-Bertani broth (Difco™), 1 L distilled water) at 30° C. and shaken cultured in a shaking incubator for one day. Each of the selected aquatic pathogenic bacteria was incubated in each specific culture medium as mentioned above for one day by the streak plate method. Then, single colony of each of the selected aquatic pathogenic bacteria was separately scrapped and inoculated in specific Luria-Bertani broths for one day.

Meanwhile, another specific culture media as mentioned above for culturing each of the selected aquatic pathogenic bacteria were prepared. Each of sterile paper disks (filter papers) was separately placed on a positioned site of each of the specific culture media, then 30 μl of Ba-BPD1 bacterial liquid was dropped on each of the sterile paper disks, at the same time, another paper disks dropped on sterile water were also separately placed on each of the specific culture media as control group. The specific culture media were then placed at room temperate 25° C. for one day. Further, the selected aquatic pathogenic bacteria liquids taken from the specific Luria-Bertani broths were separately filled in sterilized glass sprayers, appropriate volume of the selected aquatic pathogenic bacteria liquids were separately sprayed and well-distributed on the above specific culture media with paper disks dropped on Ba-BPD1 bacterial liquid and paper disks dropped on sterile water. The result of growth inhibition of the selected aquatic pathogenic bacteria in the specific culture media then was continuously observed.

Please refer to Table 3, which is the inhibition zone of B. amyloliquefaciens Ba-BPD1 to each selected aquatic pathogenic bacterium in accordance with the eleventh embodiment of the present invention. In Table 3, it was shown that the growth of the selected aquatic pathogenic bacteria could be effectively suppressed by B. amyloliquefaciens Ba-BPD1 although the result of the growth inhibition of B. amyloliquefaciens Ba-BPD1 to Vibrio parahaemolyticus would be weaker than other selected aquatic pathogenic bacteria. Accordingly, the effect of the growth inhibition of B. amyloliquefaciens Ba-BPD1 to the selected aquatic pathogenic bacteria can be proved. Meanwhile, infections of aquatic animals caused by the aquatic pathogenic bacteria could be prevented, treated or controlled by B. amyloliquefaciens Ba-BPD1. Preferably, B. amyloliquefaciens Ba-BPD1 would be able to be added into food addictive for protecting or treating aquatic animals from infections of the aquatic pathogenic bacteria.

TABLE 3
The inhibition zone of B. amyloliquefaciens Ba-BPD1 to the selected
aquatic pathogenic bacteria

| | Radius of inhibition zone¹ |
|---|---|
| bacterium | (mm) |
| Aeromonas hydrophila subsp. hydrophila | 13.64 |
| Edwardsiella tarda | 18.77 |
| Enterococcus faecalis | 6.08 |
| Enterococcus faecium | 13.45 |
| Lactococcus garvieae | 20.70 |
| Photobacterium damselae subsp. damselae | 6.91 |
| Streptococcus iniae | 15.31 |
| Vibrio parahaemolyticus | 1.18 |

¹Radius of inhibition zone (mm): the value of radius is positively correlated with the efficiency of inhibition

### Embodiment 12

### The Bacterial Inhibition Assay Between B. amyloliquefaciens Ba-BPD1 to Vibrio parahaemolyticus

To further confirm the result of the growth inhibition of B. amyloliquefaciens Ba-BPD1 to Vibrio parahaemolyticus. Another bacterial inhibition assay between B. amyloliquefaciens Ba-BPD1 to Vibrio parahaemolyticus was performed as follows.

B. amyloliquefaciens Ba-BPD1 was incubated in Luria-Bertani broth (LB, 25 g Luria-Bertani broth (Difco™), 1 L distilled water) at 30° C. and shaken cultured in a shaking incubator for one day. For explicitly observing the result of the growth inhibition of B. amyloliquefaciens Ba-BPD1 to Vibrio parahaemolyticus, Vibrio parahaemolyticus was incubated in the specific culture medium (Tryptic soy agar with 2.5% NaCl) for one day by the streak plate method. Then, Vibrio parahaemolyticus was scrapped and inoculated in a specific Luria-Bertani broth for one day.

Meanwhile, another specific culture medium (Tryptic soy agar with 2.5% NaCl) was prepared. A sterile paper disk (filter paper) was placed on a positioned site of the specific culture medium, then 30 μl of Ba-BPD1 bacterial liquid was dropped on the sterile paper disk, at the same time, another paper disk dropped on sterile water was also placed on the specific culture medium as control group. The specific culture medium was then placed at room temperate 25° C. for one day. The bacterium liquid of Vibrio parahaemolyticus taken from the specific Luria-Bertani broth was filled in sterilized glass sprayers, appropriate volume of bacterium liquid of Vibrio parahaemolyticus was sprayed and well-distributed on the specific culture medium with the paper disk dropped on Ba-BPD1 bacterial liquid and the paper disk dropped on sterile water. The result of growth inhibition of Vibrio parahaemolyticus in the specific culture medium then was continuously observed.

In accordance with the twelfth embodiment of the present invention, the result revealed that the radius of inhibition zone of B. amyloliquefaciens Ba-BPD1 to Vibrio parahaemolyticus was 8.01. It was shown that the growth of Vibrio parahaemolyticus also could be effectively suppressed by B. amyloliquefaciens Ba-BPD1 as other aquatic pathogenic bacteria as mentioned above.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A use of an isolated microorganism of Bacillus amyloliquefaciens Ba-BPD1 with Accession No.: DSM 21836 and comprising 16S ribosomal RNA comprising a nucleotide sequence of SEQ ID NO:1, for protecting or treating plants, fruit, or animals from fungal or bacterial infections.

2. The use of claim 1, wherein the infections are caused by at least one fungus or bacterium selected from the group consisting of Botrytis elliptica, Botrytis cinerea, Glomerella cingulata, Colletotrichum musae, Colletotrichum gloeosporioides, Rhizoctonia solani, Fusarium oxysporum f. sp. pisi, Fusarium oxysporum f. sp. Lycopersici, Fusarium solani, Fusarium solani, Sclerotium rolfsii Saccardo, Alternaria mali, Phytophthora capsici, Aspergillus niger, Penicillium italicum, Pestalotiopsis eugeniae, Botryodiplodia theobromae, Erwinia chrysanthemi (Erwinia carotovora subsp. carotovora), Acidovorax avenae subsp. citrulli, Agrobacterium tumefaciens, Burholderia caryophylli, Enterobactor cloaceae, Pseudomonas syringae, Ralstonia solanacearum, Xanthomonas axonopodis pv. cirti, Xanthomonas axonopodis pv. vesicatoria, Xanthomonas campestris pv. compestris, Xanthomonas oryzae pv. oryzae, Bacillus cereus, and Salmonella.

3. A use of an isolated microorganism of Bacillus amyloliquefaciens Ba-BPD1 with Accession No.: DSM 21836 and comprising 16S ribosomal RNA comprising a nucleotide sequence of SEQ ID NO:1, for an antimicrobial agent.

4. The use of claim 3, wherein the antimicrobial agent can suppress the growth of at least one microorganism selected from the group consisting of Botrytis elliptica, Botrytis cinerea, Glomerella cingulata, Colletotrichum muscle, Colletotrichum gloeosporioides, Rhizoctonia solani, Fusarium oxysporum f. sp. pisi, Fusarium oxysporum f. sp. Lycopersici, Fusarium solani, Fusarium solani, Sclerotium rolfsii Saccardo, Alternaria mali, Phytophthora capsici, Aspergillus niger, Penicillium italicum, Pestalotiopsis eugeniae, Botryodiplodia theobromae, Erwinia chrysanthemi(Erwinia carotovora subsp. carotovora), Acidovorax avenae subsp. citrulli, Agrobacterium tumefaciens, Burholderia caryophylli, Enterobactor cloaceae, Pseudomonas syringae, Ralstonia solanacearum, Xanthomonas axonopodis pv. cirti, Xanthomonas axonopodis pv. vesicatoria, Xanthomonas campestris pv. compestris, Xanthomonas oryzae pv. oryzae, Bacillus cereus, and Salmonella.

5. The use of claim 3, wherein the antimicrobial agent can suppress growth of aquatic pathogenic bacteria.

6. The use of claim 5, wherein at least one of the aquatic pathogenic bacteria is selected from the group consisting of Aeromonas hydrophila subsp. hydrophila, Edwardsiella tarda, Enterococcus faecalis, Enterococcus faecium, Lactococcus garvieae, Photobacterium damselae subsp. damselae, Streptococcus iniae, Vibrio parahaemolyticus.

7. The use of claim 1, wherein the microorganism is applied as a whole broth culture, supernatant, wettable powders, granules, water dispersible granules, suspension concentrate (flowable concentrate), flowables, or microencapsulations.

8. The use of claim 2, wherein the microorganism is applied as a whole broth culture, supernatant, wettable powders, granules, water dispersible granules, suspension concentrate (flowable concentrate), flowables, or microencapsulations.

9. The use of claim 3, wherein the microorganism is applied as a whole broth culture, supernatant, wettable powders, granules, water dispersible granules, suspension concentrate (flowable concentrate), flowables, or microencapsulations.

10. The use of claim 4, wherein the microorganism is applied as a whole broth culture, supernatant, wettable powders, granules, water dispersible granules, suspension concentrate (flowable concentrate), flowables, or microencapsulations.

11. The use of claim 5, wherein the microorganism is applied as a whole broth culture, supernatant, wettable powders, granules, water dispersible granules, suspension concentrate (flowable concentrate), flowables, or microencapsulations.

12. The use of claim 6, wherein the microorganism is applied as a whole broth culture, supernatant, wettable powders, granules, water dispersible granules, suspension concentrate (flowable concentrate), flowables, or microencapsulations.

13. The use of claim 6, wherein the antimicrobial agent is able to be added into food addictive for protecting or treating aquatic animals from infections of the aquatic pathogenic bacteria.
